(19) United States
(12) Patent Application Publication
(10) Pub. No.: US 2022/0227603 A1
(43) Pub. Date: Jul. 21, 2022

(54) Mobile Step Rail Sanitizer

(71) Applicant: Vidal Harris, Greenville, NC (US)

(72) Inventor: Vidal Harris, Greenville, NC (US)

(21) Appl. No.: 17/064,373

(22) Filed: Jan. 21, 2021

(51) Int. Cl. B66B 31/02; A61L 2/235; A61L 2/18

(52) U.S. Cl. CPC B66B 31/02; A61L 2/235; A61L 2202/16; A61L 2202/15; A61L 2/18

(57) ABSTRACT

A device particularly adapted and designed to sanitize hand rails of Escalators and Mobile Conveyors. The devices are attached to the bodies of the Escalators and Conveyors above the moving rails with the sponges touching the rails thus sanitizing them as they move under the sponges. The sponge wet with sanitizing solution should sanitize the rails and assist in the spreading of sicknesses and diseases which should give confidence to people riding them to hold on to them thus reducing the chance of injuries or death and possible insurance premium hikes.

### PRIOR APPLICATION

This application claims the benefit of U.S. Provisions of patent application Ser. No. 17/064,373 Oct. 6, 2020.

### FIELD OF THE INVENTION

This invention relates to the sanitizing of mobile step hand rails, for but not limited to Escalators, mobile belt conveyed machines found in airports. More specifically it attaches to the part of the rail coming out of the bottom and sanitizes the top of the railing as it moves with a sponge that is moisturized by a sanitization solution.

### BACKGROUND

In the Airline/Airport, mall, or any other industry that utilizes escalators or mobile belt to move individuals there are people that use man hours to clean them but can't and don't clean them continuously. Any lapse in cleaning can transmit a disease or sickness. Because of that people tend to not hold on to the rails thus not act safely which causes or can cause accidents or death which can and will raise insurance premiums.

There is a need to find a solution to constantly sanitize the railings of escalators and mobile steps and I Vidal Harris have invented the device to fulfill the need.

### SUMMARY

The Mobile Step Rail Sanitizer provides a simple way to continuously sanitize hand rails without the cost of paying man hours around the clock to continuously wipe down the rails.

The Mobile Step Rail Sanitizer is attached the to the body of the escalator or conveyer on each side (left & right) with the sponge touching the top of the rail to sanitize it as it moves under the sponge and this should offer people confidence of a clean rail to hold on to and thus be safer. The sponge will be moisten by but not limited to, a wick system, a small hole in between the solution and the sponge, or a time control dispensing mechanism or any other systematic way to keep the sponge moist. The sponge will be changed as needed and the solution will be refilled as needed.

The Mobile Step Rail Sanitizer will be attached the escalators or mobile step conveyors with brackets by way of pan head screws, suction devices, or placed in slots attached to the escalators or conveyors that the Mobile Step Rail Sanitizers fit in.

### BRIEF DESCRIPTION OF THE DRAWING

FIG. 1 is a front view of the Mobile Step Rail Sanitizer that shows the brackets that attach it to the escalator or mobile conveyor rails by way of pan head screws or suction or to the floor. FIG. 1 also shows the sanitizing solution tank with the solution under the top of the Mobile Step Rail Sanitizer that sits above the sponge to keep it moist using wicker system.

FIG. 2 is a side view of the left bracket that attaches the Mobile Step Rail Sanitizer or to the floor if the unit is made with glass and not metal. Also displays holes for the screw to go into to adjust the height.

FIG. 3 is a side view of the right bracket that attaches the Mobile Step Rail Sanitizer or to the floor if the unit is glass and not metal. Also displays holes for the screw to go into to adjust the height.

FIG. 4 is a view of the neon sanitizing symbol of the Mobile Step Rail Sanitizer that shows that sanitizing is in process for added confidence.

FIG. 5 is a front of the model with the sponge facing upward when mounted to floor with the hand sanitizer tank underneath the sponge to keep it moist using wicker system

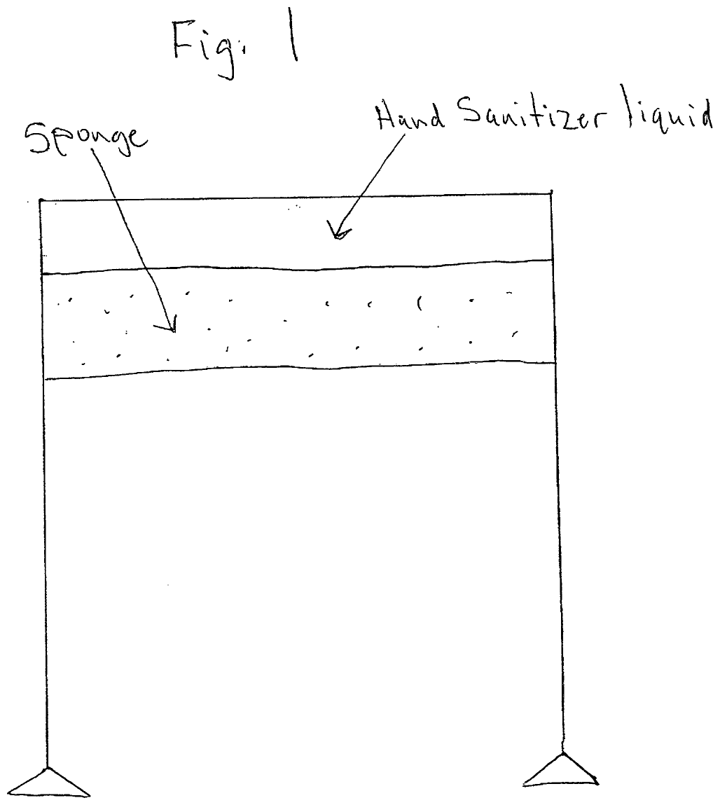

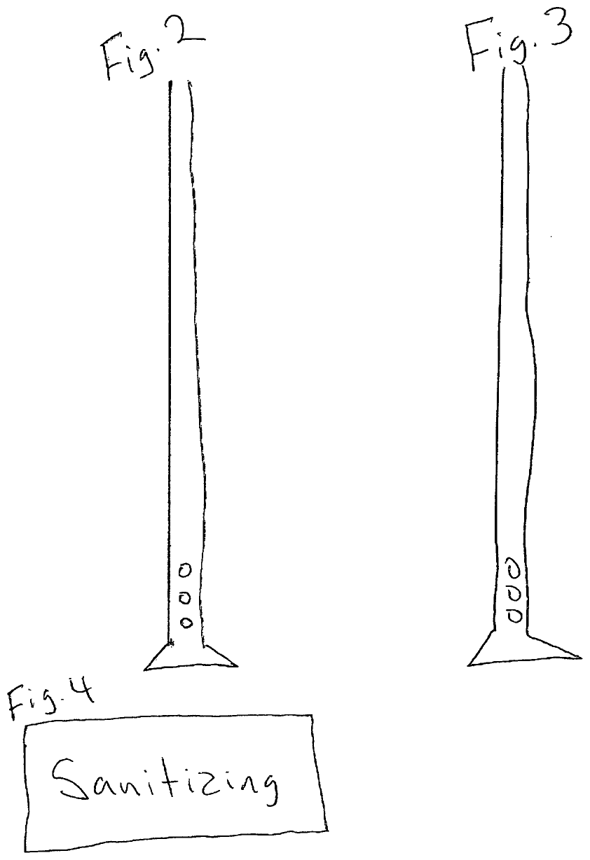

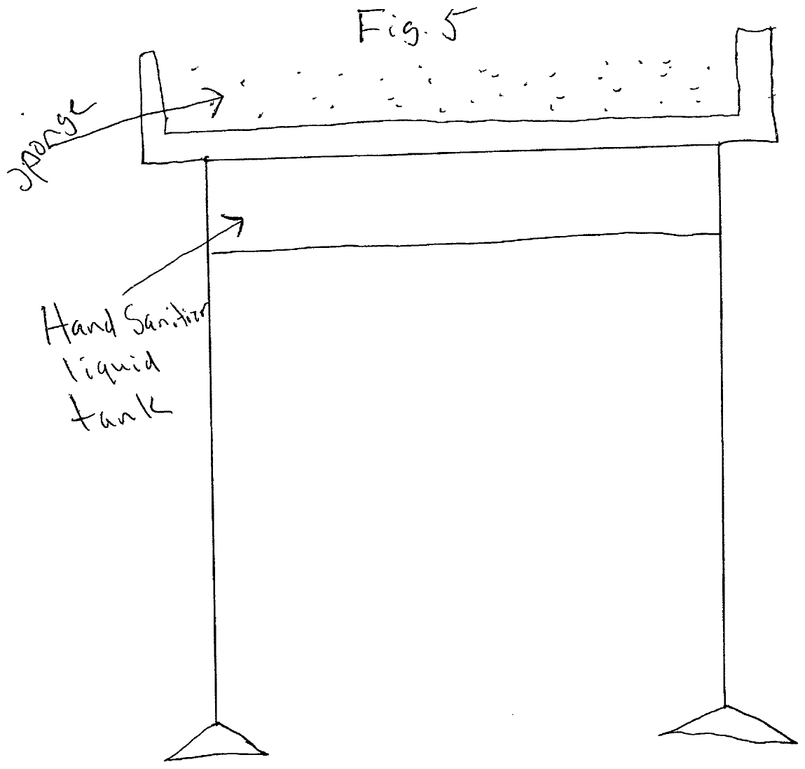

FIG. 1 is also a mirror image of how the back view looks

What is claimed is:

1. A device that attaches to Escalators or Mobile Conveyors and sanitizes the rails that people are in contact with by using a sponge that is dampen with Hand Sanitizer solution from a tank that is attached above the sponge. The tank houses the solution and keeps the sponge wet by either but not limited to a small hole, using wicker process or other method.

2. It is claimed that as the rails move under the touch of the sponge that it will be sanitized by the Mobile Step Rail Sanitizer.
